(19) United States
(12) Patent Application Publication
(10) Pub. No.: US 2005/0220873 A1
(43) Pub. Date: Oct. 6, 2005

(54) Pharmaceutical dosage forms having immediate and controlled release properties that contain a GABAB receptor agonist

(76) Inventors: Chien-Hsuan Han, Sunnyvale, CA (US); Ann F. Hsu, Los Altos Hills, CA (US); Larry Hsu, Los Altos Hills, CA (US); Charles Hsiao, Pleasanton, CA (US); Ching-Ling Diana Teng, Fremont, CA (US)

Correspondence Address: S2IPLAW, PLLC, 300 MASSACHUSETTS AVENUE, NW, SUITE 1101, WASHINGTON, DC, 20001-2692, US

(21) Appl. No.: 10/815,926

(22) Filed: Apr. 2, 2004

(51) Int. Cl. A61K 9/22

(52) U.S. Cl. 424/468

(57) ABSTRACT

The present invention relates generally to pharmaceutical dosage forms having immediate release and controlled release properties that contain a γ-aminobutyric acid (GABA_{B}) receptor agonist, e.g., baclofen, for the treatment of medical conditions, which includes spasms, cramping, and tightness of muscles, associated with ailments such as multiple sclerosis or certain spinal injuries.

### BACKGROUND OF THE INVENTION

The present invention relates generally to pharmaceutical dosage forms having immediate release and controlled release properties that contain a γ-aminobutyric acid (GABA_{B}) receptor agonist, e.g., baclofen, for the treatment of medical conditions, which includes spasms, cramping, and tightness of muscles, associated with ailments such as multiple sclerosis or certain spinal injuries.

Multiple sclerosis is considered to be an autoimmune disease. In this regard, an individual's immune system can attack the myelin sheath that surrounds nerve cells. This damage leads to muscle weakness, paralysis, poor coordination, balance problems, fatigue, and possible blindness. The GABA_{B} agonist baclofen can be used to treat these symptoms. Baclofen can also facilitate adjunct medical treatment, such as physical therapy, to improve the condition of a patient with multiple sclerosis of certain spinal injuries.

Baclofen, or 4-amino-3-(4-chlorophenyl)-butanoic acid, is a muscle relaxant and antispastic. Its mechanism of action appears unclear. Baclofen seems capable of inhibiting both monosynaptic and polysynaptic reflexes at the spinal level, possibly by hyperpolarization of afferent terminals, although actions at supraspinal sites may also occur and contribute to its clinical effect. Although baclofen is an analog of the putative inhibitory neurotransmitter GABA, there is no conclusive evidence that actions on GABA systems are involved in the production of its clinical effects. In studies with animals, baclofen has been shown to have general central nervous system (CNS) depressant properties as indicated by the production of sedation with tolerance, somnolence, ataxia, and respiratory and cardiovascular depression.

The absorption of baclofen is site specific. Baclofen is primarily absorbed in the upper gastrointestinal (GI) tract, with the extent of absorption of baclofen substantially reduced in the lower GI tract. Baclofen is rapidly and extensively absorbed. Absorption may be dose-dependent, being reduced with increasing doses. An improved method of administering baclofen to a patient would include the delivery of effective amounts of the drug to the upper GI tract for an extended period.

Several side effects are possibly associated with the administration of baclofen to mammals. These problems include nausea, vomiting, diarrhea, dizziness, daytime sedation, and less frequently, psychotic states such as depressive mood disorder. In addition, patient compliance with a dosing regimen can be suboptimal where frequent doses are required, such as the need for administering a dosage form three or four times a day. A pharmaceutical dosage form that requires less frequent dosing, such as once or twice a day, thus would be preferable. Furthermore, a pharmaceutical dosage form capable of establishing and maintaining stable plasma levels of baclofen for a prolonged period of time may benefit patients by requiring less frequent dosing and by minimizing side effects.

Certain baclofen pharmaceutical formulations, including Baclofen Tablet, 10/20 mg (Watson Pharmaceuticals, Inc., Corona, Calif.) and the orally disintegrating tablet marketed as KEMSTRO™ (Schwarz Pharma, Monheim, Germany), are marketed commercially, but do not provide controlled release of baclofen. For example, following a single 20 mg oral dose of KEMSTRO™, the peak plasma concentration is reached about 1½ hours after administration.

Various other baclofen formulations have been described. One such dosage form involves adhesive tablets placed in contact with the oral mucosa to deliver the drug across the mucous membrane. This dosage form, however, exhibits various known disadvantages associated with adhesive tablets. Furthermore, the adhesive tablets deliver baclofen to a site considered suboptimal for GABA-related agents. Other proposed baclofen formulations include a matrix dosage forms that exhibit marked swelling and high dimensional stability in the swollen state to facilitate extended gastric residence time. In addition, an osmotic pump type dosage form for delivering baclofen has also been proposed that provides for the continuous administration of drug over a prolonged period of time.

Nevertheless, there remains a significant and continuing need for, pharmaceutical dosage forms having controlled release properties that contain a GABA_{B} receptor agonist, such as baclofen, to treat medical conditions like multiple sclerosis or certain spinal injuries by establishing and maintaining stable plasma levels of the drug for a prolonged period of time to achieve less frequent dosing and to minimize side effects. These and other objectives are accomplished by the present invention.

### BRIEF SUMMARY OF THE INVENTION

The present invention relates generally to pharmaceutical dosage forms having controlled release properties that contain a GABA_{B} receptor agonist, such as baclofen. These dosage forms can be used in the treatment of medical conditions, like spasms, cramping, and tightness of muscles, which are associated with ailments such as multiple sclerosis or certain spinal injuries.

For example, the pharmaceutical dosage forms of the present invention may involve an immediate release and a controlled release component, where the immediate release component and the controlled release component each includes a GABA_{B} agonist and a pharmaceutically acceptable excipient, the immediate release component exhibits an in vitro dissolution profile comprising at least about 80% GABA_{B} agonist release after 1 hour, the controlled release component exhibits an in vitro dissolution profile in simulated intestinal fluid medium comprising at least about 5% GABA_{B} agonist release after 1 hour, at least about 20% GABA_{B} agonist release after 4 hours, and at least about 30% GABA_{B} agonist release after 6 hours, and where the ratio of the immediate release component to the controlled release component is from about 1:10 to about 10:1. The pharmaceutical dosage forms of the present invention may also involve an immediate release and a controlled release component, where the immediate release component and the controlled release component each includes a GABA_{B} agonist and a pharmaceutically acceptable excipient, the immediate release component exhibits an in vitro dissolution profile comprising at least about 80% GABA_{B} agonist release after 1 hour, where the controlled release component exhibits an in vitro dissolution profile in simulated gastric fluid/simulated intestinal fluid (1 hour switchover) medium comprising from about 2% to about 90% GABA_{B} agonist release after 1 hour, at least about 30% GABA_{B} agonist release after 4 hours, and at least about 40% GABA_{B} agonist release after 6 hours, and where the ratio of the immediate release component to the controlled release component is from about 1:10 to about 10:1. The pharmaceutical dosage forms of the present invention may also exhibit an in vivo plasma profile comprising mean maximum GABA_{B} agonist release from about 30 minutes to about 7 hours after administration to a fasting patient. Furthermore, the pharmaceutical dosage forms of the present invention may exhibit an in vivo plasma profile comprising at least 2 hours of sustained GABA_{B} agonist concentrations at greater than therapeutic levels, after about 2 hours following administration to a fasting patient.

### BRIEF DESCRIPTION OF THE SEVERAL VIEWS OF THE DRAWINGS

FIGS. 1a and 1b are graphs of the in vitro dissolution profile of a baclofen tablet formulation, 20 mg, according to measurements under the USP paddle method of 50 rpm in 900 ml simulated gastric fluid (pH 1.2) and simulated intestinal fluid (pH 6.8), respectively, at 37° C.

FIG. 2 is a graph of the in vitro dissolution profiles of baclofen tablet formulations, 20 mg, according to measurements under the USP paddle method of 50 rpm in 900 ml simulated gastric fluid (pH 1.2) at 37° C. for 1 hour with a switchover to simulated intestinal fluid (pH 6.8).

FIGS. 3a and 3b are graphs of the in vitro dissolution profile of a baclofen capsule formulation, 20 mg, according to measurements under the USP paddle method of 75 rpm in 900 ml simulated gastric fluid (pH 1.2) and simulated intestinal fluid (pH 6.8), respectively, at 37° C.

FIG. 4 is a graph of the in vitro dissolution profiles of baclofen capsule formulations, 20 mg, according to measurements under the USP paddle method of 75 rpm in 900 ml simulated gastric fluid (pH 1.2) at 37° C. for 2 hours with a switchover to simulated intestinal fluid (pH 6.8).

FIG. 5 is a graph of the in vitro dissolution profiles of baclofen capsule formulations, 30 mg, according to measurements under the USP paddle method of 75 rpm in 900 ml simulated gastric fluid (pH 1.2) at 37° C. for 2 hours with a switchover to simulated intestinal fluid (pH 6.8).

FIG. 6 is a graph of the in vivo release profiles of baclofen tablet formulations, 20 mg, where the mean baclofen plasma concentration-time profile, C_{MAX}, T_{MAX}, C_{MIN}, and T_{MIN} are determined.

FIG. 7 is a graph of the in vivo release profiles of baclofen capsule formulations, 30 mg, where the mean baclofen plasma concentration-time profile, C_{MAX}, T_{MAX}, C_{MIN}, and T_{MIN} are determined.

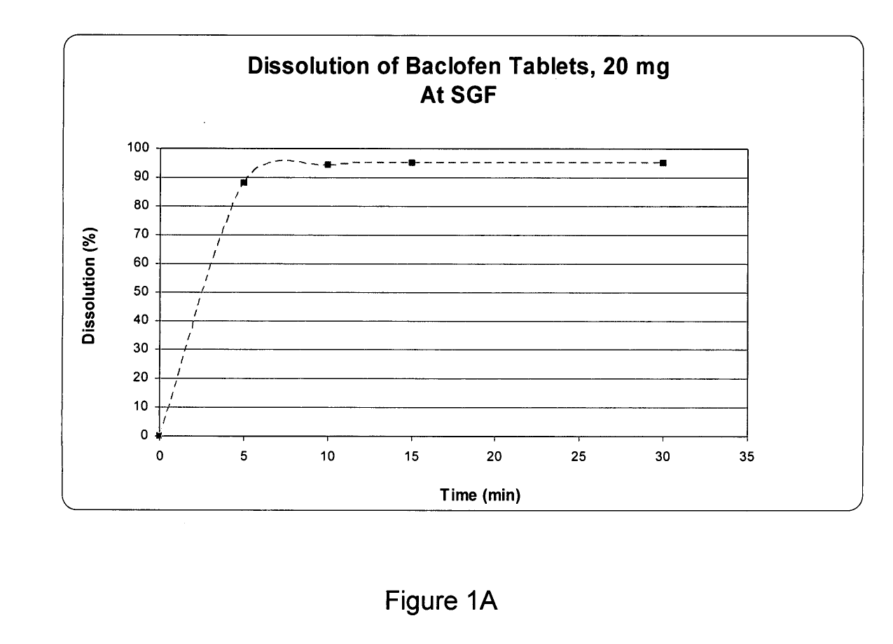

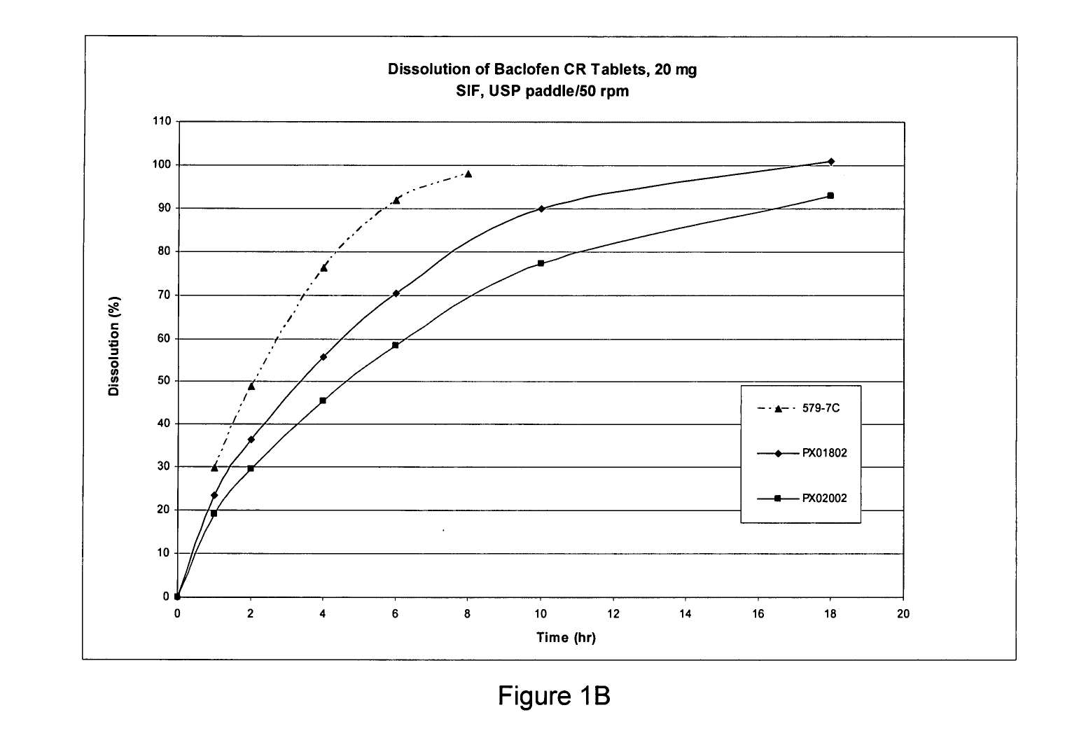

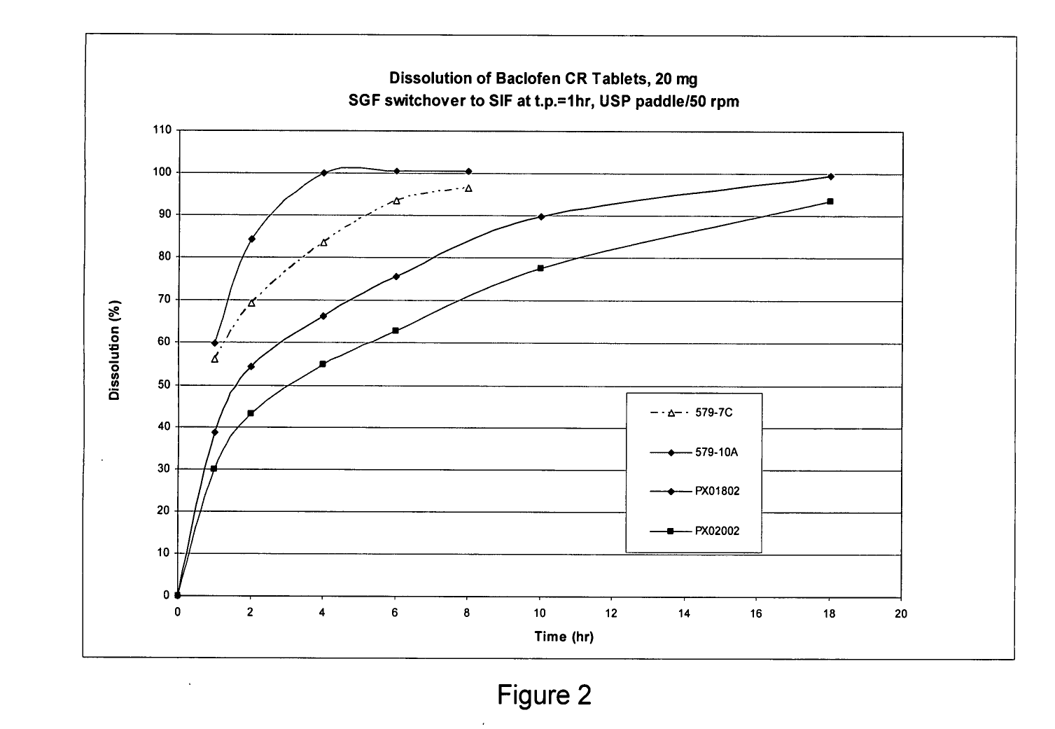

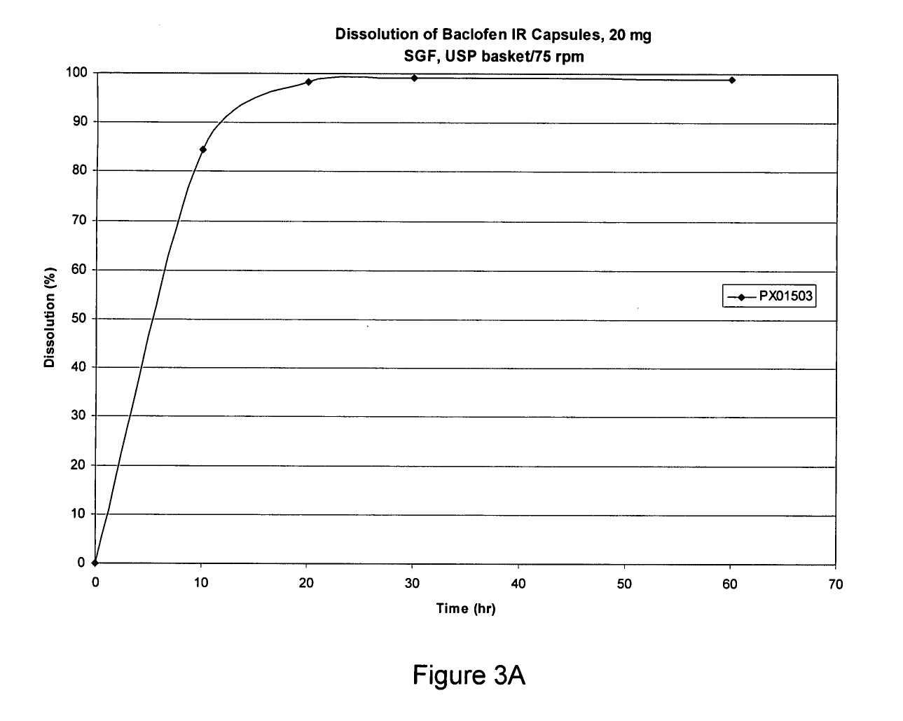

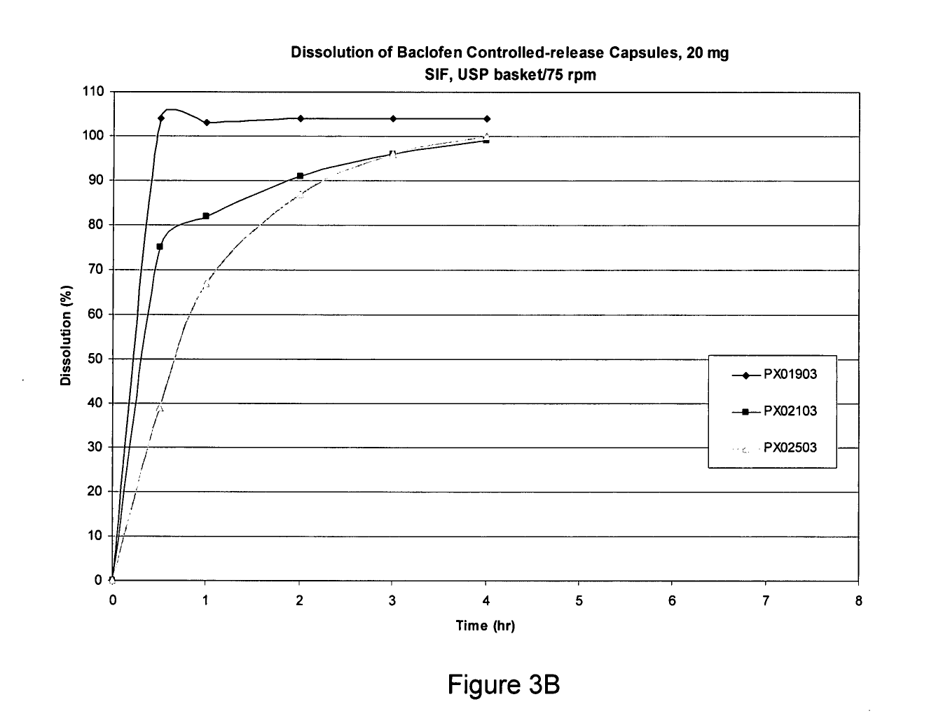

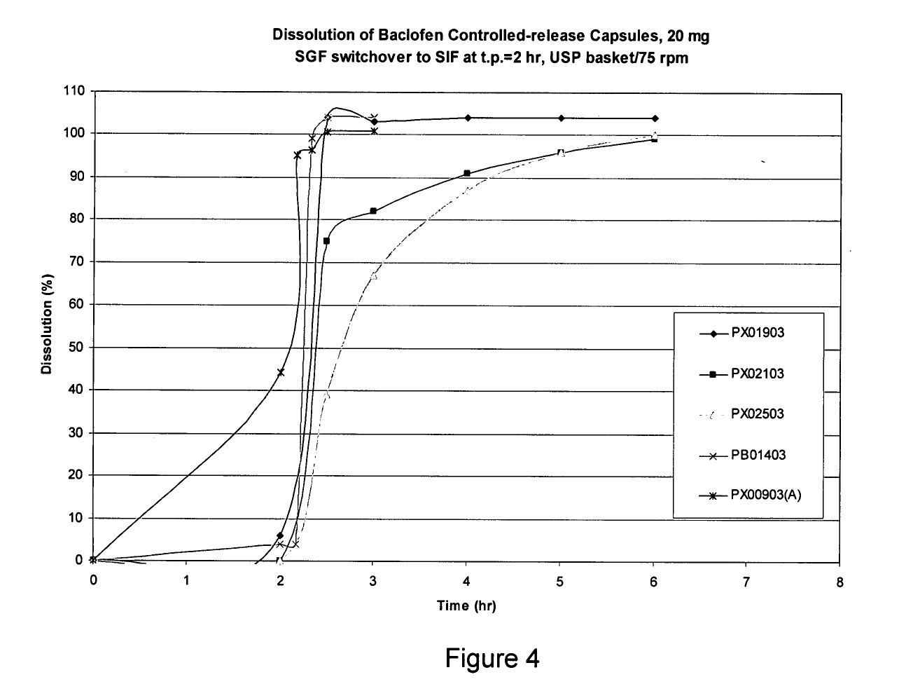

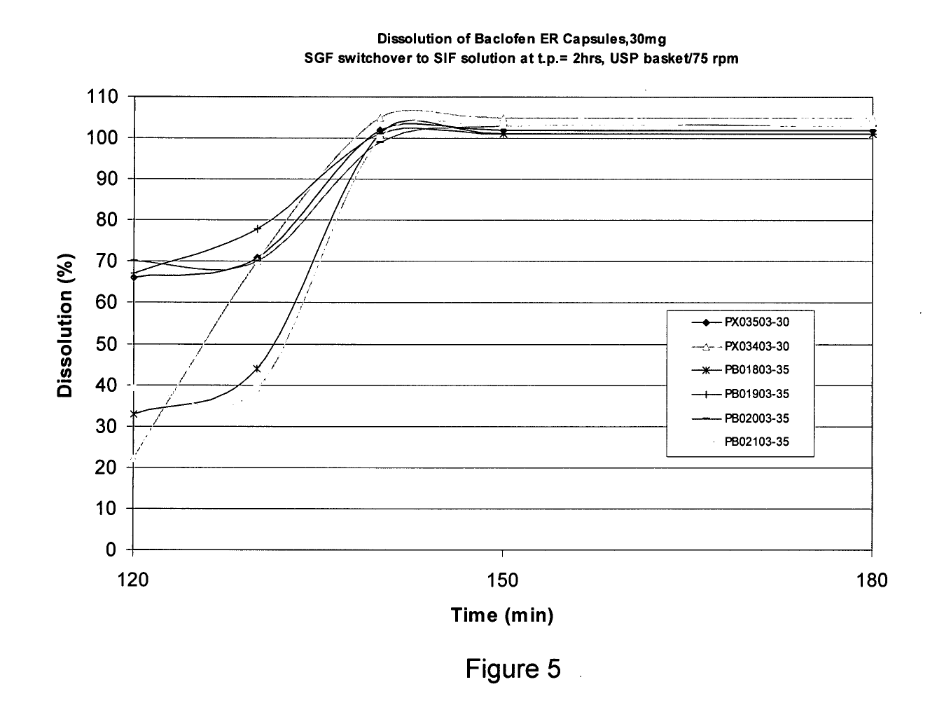

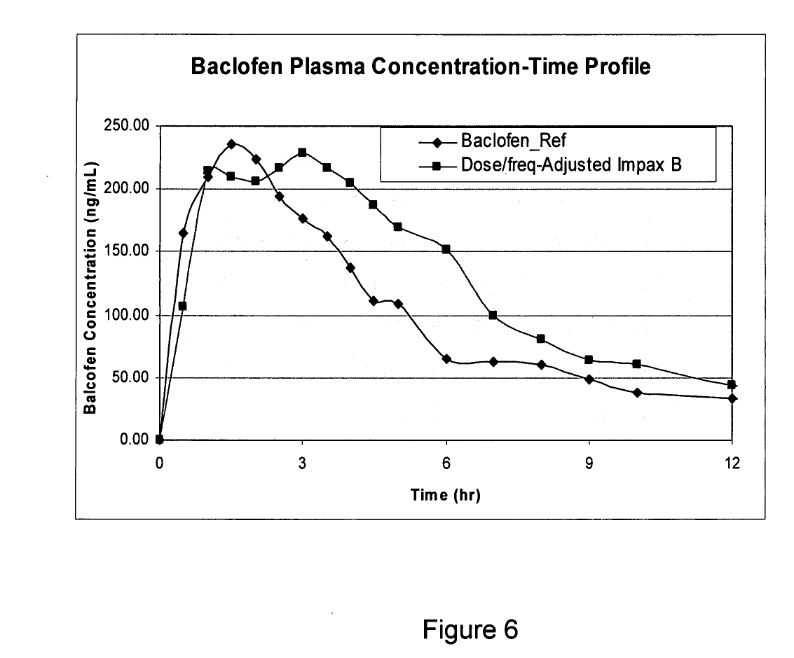

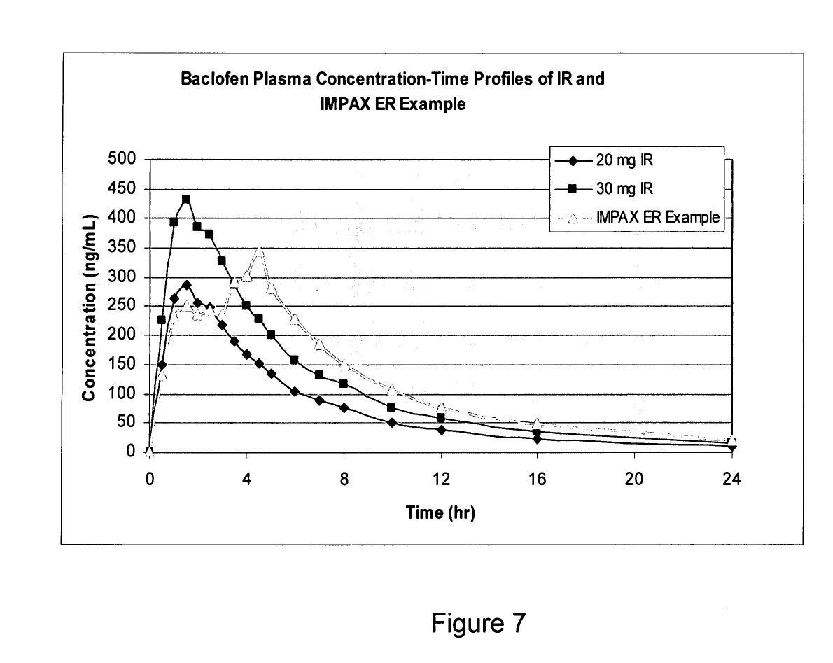

### DETAILED DESCRIPTION OF THE INVENTION

The present invention relates to pharmaceutical dosage forms comprising an immediate release and a controlled release component, wherein said immediate release component and said controlled release component each comprises a GABA_{B} agonist (preferably baclofen, a baclofen prodrug, a baclofen analog, or a mixture thereof, as well as a racemic baclofen mixture or a substantially pure L-baclofen enantiomeric product) and a pharmaceutically acceptable excipient, wherein said immediate release component exhibits an in vitro dissolution profile comprising at least about 80% GABA_{B} agonist release after 1 hour; wherein said controlled release component exhibits an in vitro dissolution profile in simulated intestinal fluid medium comprising at least about 5% GABA_{B} agonist release after 1 hour, at least about 20% GABA_{B} agonist release after 4 hours, and at least about 30% GABA_{B} agonist release after 6 hours; and wherein the ratio of said immediate release component to said controlled release component is from about 1:10 to about 10:1 (preferably about 1:4 to about 4:1, more preferably from about 1:2 to about 1:1). The pharmaceutical dosage forms of the present invention may also involve an immediate release and a controlled release component, where the immediate release component and the controlled release component each includes a GABA_{B} agonist and a pharmaceutically acceptable excipient, the immediate release component exhibits an in vitro dissolution profile comprising at least about 80% GABA_{B} agonist release after 1 hour, where the controlled release component exhibits an in vitro dissolution profile in simulated gastric fluid/simulated intestinal fluid (1 hour switchover) medium comprising from about 2% to about 90% GABA_{B} agonist release after 1 hour, at least about 30% GABA_{B} agonist release after 4 hours, and at least about 40% GABA_{B} agonist release after 6 hours, and where the ratio of the immediate release component to the controlled release component is from about 1:10 to about 10:1 (preferably about 1:4 to about 4:1, more preferably from about 1:2 to about 1:1. These dosage forms (preferably a tablet or capsule, which may contain beads, granules, particles, or a mixture thereof) may contain baclofen in the amount of from about 2 mg to about 150 mg (preferably from about 2.5 mg to about 100 mg) and can be used in the treatment of medical conditions, which includes spasms, cramping, and tightness of muscles, that are associated with ailments such as multiple sclerosis or certain spinal injuries.

An embodiment of the present invention may be a pharmaceutical dosage form comprising an immediate release and a controlled release component, wherein said immediate release component and said controlled release component each comprises a GABA_{B} agonist and a pharmaceutically acceptable excipient, and wherein said dosage form exhibits an in vivo plasma profile comprising mean maximum GABA_{B} agonist release from about 30 minutes to about 7 hours (preferably from about 1 hour to about 5.5 hours, more preferably from about 90 minutes to about 5.5 hours, and even more preferably from about 2 hours to about 5.5 hours) after administration to a fasting patient.

Another embodiment of the present invention may be a pharmaceutical dosage form comprising an immediate release and a controlled release component, wherein said immediate release component and said controlled release component each comprises a GABA_{B} agonist and a pharmaceutically acceptable excipient, and wherein said dosage form exhibits an in vivo plasma profile comprising at least 2 hours of sustained GABA_{B} agonist concentrations at greater than therapeutic levels (preferably greater than about 300 ng/ml), after about 2 hours following administration to a fasting patient. The present invention may also include embodiments in which the dosage form further comprises from about 5% to about 85% GABA_{B} agonist release in the stomach, or at least about 25% GABA_{B} agonist release in the intestinal tract, or substantially complete GABA_{B} agonist release after 10 hours following administration to a fasting patient, or a combination thereof.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms “a,” “an,” and “the” include the plural reference unless the context clearly indicates otherwise. Thus, for example, the reference to a profile is a reference to one or more such profiles, including equivalents thereof known to those skilled in the art. Other than in the operating examples, or where otherwise indicated, all numbers expressing quantities of ingredients or reaction conditions used herein should be understood as modified in all instances by the term “about.” The term “about” when used in connection with percentages can mean ±1%.

All patents and other publications identified are incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason.

Unless defined otherwise, all technical and scientific terms used herein have the same meaning as those commonly understood to one of ordinary skill in the art to which this invention pertains. Although any known methods, devices, and materials may be used in the practice or testing of the invention, the preferred methods, devices, and materials in this regard are described here.

The baclofen, also known as butanoic acid or 4-amino-3-(4-chlorophenyl) butanoic acid, of the present invention includes racemic baclofen, enantiomerically pure L-baclofen, and analogs, derivatives, prodrugs, metabolites thereof, and any pharmaceutically acceptable salts thereof.

Baclofen is a GABA_{B} receptor agonist, and thus other GABA_{B} receptor agonists are envisioned within the scope of the invention. These may include 4-aminobutanoic acid (GABA); 3-aminopropyl)methylphosphinic acid; 4-amino-3-phenylbutanoic acid; 4-amino-3-hydroxybutanoic acid; 4-amino-3-(4-chlorophenyl)-3-hydroxyphenylbutanoic acid; 4-amino-3-(thien-2-yl)butanoic acid; 4-amino-3-(5-chlorothien-2-yl)butanoic acid; 4-amino-3-(5-bromothien-2-yl)butanoic acid; 4-amino-3-(5-methylthien-2-yl)butanoic acid; 4-amino-3-(2-imidazolyl)butanoic acid; 4-guanidino-3-(4-chlorophenyl)butanoic acid; 3-amino-2-(4-chlorophenyl)-1-nitropropane; (3-aminopropyl)phosphonous acid; (4-aminobut-2-yl)phosphonous acid; (3-amino-2-methylpropyl)phosphonous acid; (3-aminobutyl)phosphonous acid; (3-amino-2-(4-chlorophenyl)propyl)phosphonous acid; (3-amino-2-(4-chlorophenyl)-2-hydroxypropyl)phosphonous acid; (3-amino-2-(4-fluorophenyl)propyl)phosphonous acid; (3-amino-2-phenylpropyl)phosphonous acid; (3-amino-2-hydroxypropyl)phosphonous acid; (E)-(3-aminopropen-1-yl)phosphonous acid; (3-amino-2-cyclohexylpropyl)phosphonous acid; (3-amino-2-benzylpropyl)phosphonous acid; [3-amino-2-(4-methylphenyl)propyl]phosphonous acid; [3-amino-2-(4-trifluoromethylphenyl)propyl]phosphonous acid; [3-amino-2-(4-methoxyphenyl)propyl]phosphonous acid; [3-amino-2-(4-chlorophenyl)-2-hydroxypropyl]phosphonous acid; (3-amino propyl)methylphosphinic acid; (3-amino-2-hydroxypropyl)methylphosphinic acid; (3-aminopropyl)(difluoromethyl)phosphinic acid; (4-aminobut-2-yl)methylphosphinic acid; (3-amino-1-hydroxypropyl)methylphosphinic acid; (3-amino-2-hydroxypropyl)(difluoromethyl)phosphinic acid; (E)-(3-aminopropen-1-yl)methylphosphinic acid; (3-amino-2-oxo-propyl)methyl phosphinic acid; (3-aminopropyl)hydroxymethylphosphinic acid; (5-aminopent-3-yl)methylphosphinic acid; (4-amino-1,1,1-trifluorobut-2-yl)methylphosphinic acid; (3-amino-2-(4-chlorophenyl)propyl)sulfinic acid; 3-aminopropylsulfinic acid, 1-(aminomethyl)cyclohexaneacetic acid, and the like. See, e.g., U.S. Pat. No. 6,664,069.

For purposes of the present invention the term “GABA related active agents” refers to all of those active agents referred to in U.S. Pat. No. 6,350,769, issued Feb. 26, 2002, to Kaufman et al., fully incorporated herein by reference.

The term “analog” means a compound which comprises a chemically modified form of a specific compound or class thereof, and which maintains the pharmaceutical and/or pharmacological activities characteristic of said compound or class. For example, baclofen analogs include 3-thienyl- and 3-furylaminobutyric acids.

The term “derivative” means a chemically modified compound wherein the modification is considered routine by the ordinary skilled chemist, such as an ester or an amide of an acid, protecting groups, such as a benzyl group for an alcohol or thiol, and tert-butoxycarbonyl group for an amine.

The term “prodrug”, as used herein, includes any covalently bonded carriers which release an active parent drug of the present invention in vivo when such prodrug is administered to a patient. Because prodrugs are known to enhance numerous desirable qualities of pharmaceuticals (i.e., solubility, bioavailability, manufacturing, etc.) the compounds of the present invention may be delivered in prodrug form. Prodrugs of the present invention may be prepared by modifying functional groups present in the compound in such a way that the modifications are cleaved, either in routine manipulation or in vivo, to the parent compound. The transformation in vivo may be, for example, as the result of some metabolic process, such as chemical or enzymatic hydrolysis of a carboxylic, phosphoric or sulphate ester, or reduction or oxidation of a susceptible functionality. Prodrugs within the scope of the present invention include compounds wherein a hydroxy, amino, or sulfhydryl group is bonded to any group that, when the prodrug of the present invention is administered to a mammalian subject, it cleaves to form a free hydroxyl, free amino, or free sulfhydryl group, respectively. Functional groups that may be rapidly transformed, by metabolic cleavage, in vivo form a class of groups reactive with the carboxyl group of the compounds of this invention. They include, but are not limited to such groups as alkanoyl (such as acetyl, propionyl, butyryl, and the like), unsubstituted and substituted aroyl (such as benzoyl and substituted benzoyl), alkoxycarbonyl (such as ethoxycarbonyl), trialkysilyl (such as trimethyl- and triethysilyl), monoesters formed with dicarboxylic acids (such as succinyl), and the like. Because of the ease with which metabolically cleavable groups of the compounds useful according to this invention are cleaved in vivo, the compounds bearing such groups act as prodrugs. The compounds bearing the metabolically cleavable groups have the advantage that they may exhibit improved bioavailability as a result of enhanced solubility and/or rate of absorption conferred upon the parent compound by virtue of the presence of the metabolically cleavable group.

A discussion of prodrugs is provided in the following: DESIGN OF PRODRUGS, H. Bundgaard, ed. (Elsevier, 1985); METHODS IN ENZYMOLOGY, K. Widder et al., eds., vol. 42, 309-96 (Academic Press 1985); A TEXTBOOK OF DRUG DESIGN AND DEVELOPMENT, Krogsgaard-Larsen & H. Bundgaard, ed., Chapter 5; Design and Applications of Prodrugs, 113-91 (1991); H. Bundgard, Advanced Drug Delivery Reviews, 1-38 (1992); 8 J. PHARM. SCIENCES 285 (1988); N. Nakeya et al., 32 CHEM. PHARM. BULL. 692 (1984); T. Higuchi and V. Stella, Prodrugs as Novel Delivery Systems, 14 A.C.S. SYMPOSIUM SERIES: BIOREVERSIBLE CARRIERS IN DRUG DESIGN, Edward B. Roche, ed. (Am. Pharm. Assoc. & Pergamon Press 1987), each of which is incorporated herein by reference.

Thus, the present invention contemplates the use of prodrugs of GABA_{B} receptor agonists (including baclofen), methods of delivering the same, and compositions containing the same. For example, baclofen prodrugs have been described in Leisen et al., Lipophilicities of Baclofen Ester Prodrugs Correlate with Affinities to the ATP-dependent Efflux Pump P-glycoprotein, 20 PHARM. RES. 772-78 (2003).

The term “metabolite” refers to a form of a compound obtained in a human or animal body by action of the body on the administered form of the compound, for example a de-methylated analog of a compound bearing a methyl group which is obtained in the body after administration of the methylated compound as a result of action by the body on the methylated compound. Metabolites may themselves have biological activity.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

For example, “pharmaceutically acceptable salts” refer to derivatives of the disclosed compounds wherein the specified compound is converted to an acid or base salt thereof. Such pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluensulfonic, methanesulfonic, ethane dislfonic, oxalic, isethionic, and the like.

For purposes of the present invention the term “controlled release” refers to part of all of a dosage form that can release one or more active pharmaceutical agents over a prolonged period of time (i.e., over a period of more than 1 hour). The characteristic of controlled release (CR) may also be referred to as sustained release (SR), prolonged release (PR), or extended release (ER). When used in association with the dissolution profiles discussed herein, the term “controlled release” refers to that portion of a dosage form according to the present invention that delivers active agent over a period of time greater than 1 hour.

“Immediate release” refers to part or all of a dosage form that releases active agent substantially immediately upon contact with gastric juices and that results in substantially complete dissolution within about 1 hour. The characteristic of immediate release (IR) may also be referred to as instant release (IR). When used in association with the dissolution profiles discussed herein, the term “immediate release” refers to that portion of a dosage form according to the present invention that delivers active agent over a period of time less than 1 hour.

Initial peak plasma level refers to the first rise in blood plasma level of the active agent and may be followed by one or more additional peaks, one of which may be referred to as C_{MAX}. “C” is shorthand for concentration, “T” for time, “max” for maximum, and “min” for minimum. The term “C_{MAX}” is the peak blood plasma concentration exhibited by the compositions of the present invention. “T_{MAX}” refers to the time that C_{MAX} occurs in the plasma concentration-time profile. “C_{MIN}” is the minimum plasma concentration and “T_{MIN}” is the time that C_{MIN} occurs. Initial peak plasma level refers to the first rise in blood plasma level of the active agent and may be followed by one or more additional peaks, one of which may be C_{MAX}. As used herein, “mean maximum GABA_{B} agonist release” refers to the mean GABA_{B} agonist C_{MAX}.

The USP paddle method refers to the Paddle and Basket Method as described in United States Pharmacopoeia, Edition XXII (1990). In particular, the USP paddle method of 50 rpm or 75 rpm in 900 ml SGF or SIF at pH 1.2 or pH 6.8 at 37° C. may be used to determine the in vitro dissolution profiles according to the present invention.

As used herein, the term “patient” means any mammal including humans.

The term “effective amount” means an amount of a compound/composition according to the present invention effective in producing the desired therapeutic effect.

Total daily dosages of the compounds useful according to this invention administered to a host in single or divided doses are generally in amounts of from about 0.01 mg/kg to about 100 mg/kg body weight daily, and preferably from about 0.05 mg/kg to about 50 mg/kg body weight daily. It should be understood, however, that the specific dose level for any particular patient will depend upon a variety of factors including body weight, general health, gender, diet, time and route of administration, rates of absorption and excretion, combination with other drugs, and the severity of the particular disease being treated. Actual dosage levels of active ingredient in the compositions of the present invention may be varied so as to obtain an amount of active ingredient that is effective to obtain a desired therapeutic response for a particular composition and method of administration. The selected dosage level, therefore, depends upon the desired therapeutic effect, on the route of administration, on the desired duration of treatment, and other factors. Total daily dose of the compounds useful according to this invention administered to a host in single or divided doses may be in amounts, for example, of from about 0.01 mg/kg to about 20 mg/kg body weight daily and preferably 0.02 to 10 mg/kg/day. The preferred dosage range of baclofen is between 2.5 mg and 100 mg per dosage form. Dosage forms according to the present invention may contain such amounts or fractions thereof as may be used to make up the daily dose.

“Mean plasma concentration-time profile” is the mathematical average of plasma concentration at each time point over a 24-hr period obtained in at least 12 healthy adult male and female subjects. Sampling times are 0.5, 1, 1.5, 2, 2.5, 3, 3.5, 4, 4.5, 5, 6, 7, 8, 10, 12, 16, and 24 hours.

The term “effective amount” means an amount of a compound/composition according to the present invention effective in producing the desired therapeutic effect.

The term “excipients” refer to pharmacologically inert ingredients that are not active in the body. See HANDBOOK OF PHARMACEUTICAL EXCIPIENTS (Am. Pharm. Ass'n 1986). The artisan of ordinary skill in the art will recognize that many different excipients can be used in formulations according to the present invention and the list provided herein is not exhaustive.

The active ingredients of the present invention may be mixed with pharmaceutically acceptable carriers, diluents, adjuvants, excipients, or vehicles, such as preserving agents, fillers, polymers, disintegrating agents, glidants, wetting agents, emulsifying agents, suspending agents, sweetening agents, flavoring agents, perfuming agents, lubricating agents, acidifying agents, and dispensing agents, depending on the nature of the mode of administration and dosage forms. Such ingredients, including pharmaceutically acceptable carriers and excipients that may be used to formulate oral dosage forms. Pharmaceutically acceptable carriers include water, ethanol, polyols, vegetable oils, fats, waxes polymers, including gel forming and non-gel forming polymers, and suitable mixtures thereof. Examples of excipients include starch, pregelatinized starch, Avicel, lactose, milk sugar, sodium citrate, calcium carbonate, dicalcium phosphate, and lake blend. Examples of disintegrating agents include starch, alginic acids, and certain complex silicates. Examples of lubricants include magnesium stearate, sodium lauryl sulphate, talc, as well as high molecular weight polyethylene glycols.

“Dosing under fasting conditions” is defined as when the dosage is administered orally with 240 ml of room temperature water after subjects are fasted overnight for at least 10 hours. No fluid, except that given with drug administration, will be allowed from 1 hour prior to dose administration until 1 hour after dosing. At 2 hours post-dose, subjects will consume 240 ml of room temperature water.

“Fasting” is defined as: A light snack will be served approximately 10 hours prior to dose administration after which a fast (except water) will be maintained until at least 4 hours after dosing. Clear fluids, such as water, will be allowed during fasting as described above.

The term “released in the stomach” means released at a pH consistent with the pH in a patients stomach. The rate and amount of release in the stomach may be ascertained in vitro using standard USP dissolution test or in vivo using actual patient studies.

The term “released in the intestine” means at a pH consistent with the pH in a patient's small intestine. The rate and amount of release in the intestine may be ascertained in vitro using standard USP dissolution test or in vivo using actual patient studies.

“After administration” refers to the time after the patient or study subject has taken, by oral administration, the baclofen-containing formulation.

“In vitro” refers to testing done outside of a patient's body, for example in special laboratory apparatus. For example, standard USP dissolution tests are known in the art and taught, for example, by the United States Pharmacopoeia, Edition XXII (1990). These include, for example, testing baclofen-containing formulations at 50 rpm or 75 rpm in 900 ml SGF or SIF at pH 1.2 or pH 6.8 at 37° C.

“In vivo” refers to testing performed in a subject's or patient's body.

“Steady state” refers to the repeated dosing of a drug until it reaches a stable level of absorption and elimination such that the amount of drug in the body is constant.

An object of the present invention provides for controlled release baclofen compositions having improved plasma concentration-time profiles. Various controlled release baclofen compositions have been reported. For example, U.S. Pat. No. 5,091,184, issued Feb. 25, 1992, to Khanna describes adhesive tablets that stick to the oral mucosa and deliver drug through the mucous membrane. These compositions have one or more of the problems associated with adhesive tablets and deliver the drug to a less than optimal site for GABA related drugs. Additionally, U.S. Pat. No. 5,651,985, issued Jul. 29, 1997, to Penners et al. refers to matrix dosage forms having extended gastric residence time. Dosage forms made according to the Penners reference are described as having marked swelling and high dimensional stability in the swollen state. In addition, an osmotic pump type dosage form (a hydrogel containing tiny pills) for delivering baclofen is referred to in U.S. Pat. No. 4,764,380, issued Aug. 16, 1988, to Urquhart et al., which describes the continuous administration of drug over a prolonged period of time.

One embodiment of the present invention provides a controlled release solid oral dosage form in which there is immediate release of baclofen and delayed or delayed-sustained release of baclofen. Dosages according to the present invention may include an immediate release component and a delayed or delayed-sustained release component. The combination of these two components can release the drug in a pulsed release fashion or a continuous fashion upon oral administration of the dosage form.

The delayed and delayed-sustained release component delays the release of the drug for a specified time period, at which time the release of the drug may be in a pulsed fashion, i.e., the dose may be released within about a 1- to 30-minute interval or less than an hour, or the release may be a continuous sustained release, i.e., the drug is released over a period of up to 7 hours.

In one aspect, the invention relates to a controlled release baclofen solid oral dosage form comprising an immediate release baclofen component and a delayed or delayed-sustained, or sustained release baclofen component. The immediate release baclofen component comprises baclofen formulated with one or more pharmaceutically acceptable excipients that allow for immediate release of the baclofen, and the delayed, or delayed-sustained, or sustained release baclofen component comprises baclofen formulated with one or more excipients that allow for delayed, or delayed-sustained, or sustained release of the baclofen. For example, see U.S. Pat. No. 6,372,254 that refers to formulations, such as tablets, having both an immediate release component and an extended release component.

Among other dosage forms apparent to the skilled artisan the solid oral dosage form according to the present invention may be a tablet formulation, or a discrete unit-filled capsule formulation, or a sachet formulation. The discrete units of the present invention include beads, granules, pellets, spheroids, particles, tablets, pills, etc.

Specifically, the immediate release, delayed release, delayed-sustained release, and sustained release components of the dosage form can take any form known to a skilled pharmaceutical formulator, including one component of a multi-component tablet such as described in U.S. Pat. 6,372,254, issued Apr. 16, 2002, and pending U.S. patent applications Ser. No. 10/241,837, filed Sep. 12, 2002, and WO 03101432, filed Dec. 11, 2003, each assigned to Impax Laboratories, Inc. The controlled release baclofen dosages according to the present invention may be in the form of cores comprising baclofen.

Dosage forms can be made according to known methods in the art. Some preferred methods are described below.

Matrix Dosage Forms. The term matrix, as used herein, refers to a solid material having an active agent incorporated therein. Upon exposure to a dissolution media, channels are formed in the solid material so that the active agent can escape. Dosage forms according to one embodiment of the present invention may be in the form of coated or uncoated matrices. A coating, for example may contain immediate release baclofen, or in the alternative, and the matrix itself can contain controlled release baclofen. Drug release from the delayed or delayed-sustained, or sustained release component can be immediate or sustained, for example within 7 hours after oral administration of the oral dosage form to ensure effective absorption of the drug.

The delayed release baclofen component may be comprised of baclofen coated with at least one delayed release layer. The delayed-sustained release baclofen component may be comprised of sustained-release-coated baclofen coated with at least one delayed release layer. The sustained release baclofen component may be comprised of baclofen coated with at least one sustained-release polymer, or a matrix-controlled release polymer.

The skilled artisan should appreciate that the matrix material can be chosen from a wide variety of materials that can provide the desired dissolution profiles. Materials can include, for example, one or more gel forming polymers such as polyvinyl alcohol, cellulose ethers including, for example, hydroxyl propyl alkyl, celluloses such as hydroxypropyl methyl cellulose, hydroxy alkyl celluloses such as hydroxy propyl cellulose, natural or synthetic gums such as guar gum, xanthum gum, and alginates, as well as, ethyl cellulose, polyethylene oxide, polyvinyl pyrrolidone, fats, waxes, polycarboxylic acids or esters such as the Carbopol® series of polymers, methacrylic acid copolymers, and methacrylate polymers.

Methods of making matrix dosages are known in the art and any such method that can yield the desired immediate release and controlled release dissolution profiles may be relied upon according to the present invention. One such method involves baclofen with a solid polymeric material and one or more pharmaceutically acceptable excipients that are then blended and compressed in controlled release tablet cores. Such tablet cores can be used for further processing as bilayer tablets, press coated tablets, or film coated tablets.

A coating containing the immediate release baclofen can be added to the outside of the controlled release tablet cores to produce a final dosage form. Such a coating can be prepared by mixing baclofen with polyvinylpyrrolidone (PVP) 29/32 or hydroxypropyl methylcellulose (HPMC) and water/isopropyl alcohol and triethyl acetate. Such an immediate release coating can be spray coated onto the tablet cores. The immediate release coating may also be applied using a press-coating process with a blend consisting of 80% by weight baclofen and 20% by weight of lactose and hydroxypropyl methylcellulose type 2910. Press coating techniques are known in the art and are described in U.S. Pat. No. 6,372,254 (Ting et al.), incorporated herein by reference in its entirety.

In addition, the formulation of respective release components can occur by appropriate granulation methods as is well known in the art. In wet granulation, solutions of the binding agent (polymer) are added with stirring to the mixed powders. The powder mass is wetted with the binding solution until the mass has the consistency of damp snow or brown sugar. The wet granulated material is forced through a sieving device. Moist material from the milling step is dried by placing it in a temperature controlled container. After drying, the granulated material is reduced in particle size by passing it through a sieving device. Lubricant is added, and the final blend is then compressed into a matrix dosage form.

In fluid-bed granulation, particles of inert material and/or active agent are suspended in a vertical column with a rising air stream. While the particles are suspended, a common granulating material in solution is sprayed into the column. There is a gradual particle buildup under a controlled set of conditions resulting in tablet granulation. Following drying and the addition of lubricant, the granulated material is ready for compression.

In dry-granulation, the active agent, binder, diluent, and lubricant are blended and compressed into tablets. The compressed large tablets are comminuted through the desirable mesh screen by sieving equipment. Additional lubricant is added to the granulated material and blended gently. The material is then compressed into tablets.

Particle Based Dosage Forms, Immediate Release Particles. The immediate release/controlled release dosage forms of the present invention can also take the form of pharmaceutical particles. The dosage forms can include immediate release particles in combination with controlled release particles in a ratio sufficient to deliver the desired dosages of active agents. The controlled release particles can be produced by coating the immediate release particles.

The particles can be produced according to any of a number of known methods for making particles. The immediate release particles comprise the active agent combination and a disintegrant. Suitable disintegrants include, for example, starch, low-substitution hydroxypropyl cellulose, croscarmellose sodium, calcium carboxymethyl cellulose, hydroxypropyl starch, sodium starch glycolate, and microcrystalline cellulose.

In addition to the above-mentioned ingredients, the matrix may also contain suitable quantities of other materials, for example, diluents, lubricants, binders, granulating aids, colorants, flavorants, and glidants that are conventional in the pharmaceutical arts. The quantities of these additional materials are sufficient to provide the desired effect to the desired formulation. A matrix incorporating particles may also contain suitable quantities of these other materials such as diluents, lubricants, binders, granulating aids, colorants, flavorants, and glidants that are conventional in the pharmaceutical arts in amounts up to about 75% by weight of the particulate, if desired.

The term “particle” as used herein means a granule having a diameter of between about 0.01 mm and about 5.0 mm, preferably between about 0.1 mm and about 2.5 mm, and more preferably between about 0.5 mm and about 2 mm. The skilled artisan should appreciate that particles according to the present invention can be any geometrical shape within this size range and so long as the mean for a statistical distribution of particles falls within the particle sizes enumerated above, they will be considered to fall within the contemplated scope of the present invention. Particles can assume any standard structure known in the pharmaceutical arts. Such structures include, for example, matrix particles, non-pareil cores having a drug layer and active or inactive cores having multiple layers thereon. A controlled release coating can be added to any of these structures to create a controlled release particle.

In one preferred embodiment, oral dosage forms are prepared to include an effective amount of particles as described above within a capsule. For example, melt-extruded particles may be placed in a gelatin capsule in an amount sufficient to provide an effective controlled release dose when ingested and contacted by gastric fluid. In another preferred embodiment, a suitable amount of the particles are compressed into an oral tablet using conventional tableting equipment using standard techniques. Techniques and compositions for making tablets (compressed and molded), capsules (hard and soft gelatin), and pills are also described in REMINGTON'S PHARMACEUTICAL SCIENCES, Arthur Osol, ed., 1553-93 (1980), incorporated herein by reference. The particles can be made by mixing the relevant ingredients and granulating the mixture. The resulting particles are dried and screened, and the particles having the desired size are used for drug formulation.

Controlled Release Particles. The controlled release particles of the present invention slowly release baclofen when ingested and exposed to gastric fluids, and then to intestinal fluids. The controlled release profile of the formulations of the invention can be altered, for example, by increasing or decreasing the thickness of the retardant coating, i.e., by varying the amount of overcoating. The resultant solid controlled release particles may thereafter be placed in a gelatin capsule in an amount sufficient to provide an effective controlled release dose when ingested and contacted by an environmental fluid, e.g., gastric fluid, intestinal fluid or dissolution media. The particles may be overcoated with an aqueous dispersion of a hydrophobic or hydrophilic material to modify the release profile. The aqueous dispersion of hydrophobic material preferably further includes an effective amount of plasticizer, e.g. triethyl citrate. Preformulated aqueous dispersions of ethylcellulose, such as Aquacoat® or Surelease®, may be used. If Surelease® is used, it is not necessary to separately add a plasticizer.

The release of the therapeutically active agent from the controlled release formulation of the present invention can be further influenced, i.e., adjusted to a desired rate, by the addition of one or more release-modifying agents. The release-modifying agent may be organic or inorganic and include materials that can be dissolved, extracted, or leached from the coating in the environment of use. The pore-formers may comprise one or more hydrophilic materials such as hydroxypropyl methylcellulose. The release-modifying agent may also comprise a semi-permeable polymer. In certain preferred embodiments, the release-modifying agent is selected from hydroxypropyl methylcellulose, lactose, metal stearates, and mixtures thereof.

The controlled-release component may also include a combination of hydrophilic and hydrophobic polymers. In this embodiment, once administered, the hydrophilic polymer dissolves away to weaken the structure of the controlled-release component, and the hydrophobic polymer retards the water penetration and helps to maintain the shape of the drug delivery system.

The hydrophobic material may be selected from the group consisting of alkylcellulose, acrylic and methacrylic acid polymers and copolymers, shellac, zein, hydrogenated castor oil, hydrogenated vegetable oil, or mixtures thereof. In certain preferred embodiments, the hydrophobic material is a pharmaceutically acceptable acrylic polymer, including but not limited to acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer, poly(methyl methacrylate), poly(methacrylic acid anhydride), polymethacrylate, polyacrylamide, poly(methacrylic acid anhydride), and glycidyl methacrylate copolymers. In alternate embodiments, the hydrophobic material is selected from materials such as one or more hydroxyalkyl celluloses such as hydroxypropyl methycellulose. The hydroxyalkyl cellulose is preferably a hydroxy (C₁ to C₆) alkyl cellulose, such as hydroxypropylcellulose, hydroxypropylmethylcellulose, or preferably hydroxyethylcellulose. The amount of the hydroxyalkyl cellulose in the present oral dosage form is determined, inter alia, by the precise rate of active agents desired and may vary from about 1% to about 80%.

In embodiments of the present invention where the coating comprises an aqueous dispersion of a hydrophobic polymer, the inclusion of an effective amount of a plasticizer in the aqueous dispersion of hydrophobic polymer can further improve the physical properties of the film. For example, because ethylcellulose has a relatively high glass transition temperature and does not form flexible films under normal coating conditions, it is necessary to plasticize the ethylcellulose before using it as a coating material. Generally, the amount of plasticizer included in a coating solution is based on the concentration of the film-former, e.g., most often from about 1 percent to about 50 percent by weight of the film-former. Concentration of the plasticizer, however, is preferably determined after careful experimentation with the particular coating solution and method of application.

Examples of suitable plasticizers for ethylcellulose include water-insoluble plasticizers such as dibutyl sebacate, diethyl phthalate, triethyl citrate, tributyl citrate, and triacetin, although other water-insoluble plasticizers (such as acetylated monoglycerides, phthalate esters, castor oil, etc.) may be used. Triethyl citrate is an especially preferred plasticizer for the aqueous dispersions of ethyl cellulose of the present invention.

Examples of suitable plasticizers for the acrylic polymers of the present invention include, but are not limited to, citric acid esters such as triethyl citrate NF XVI, tributyl citrate, dibutyl phthalate, and possibly 1,2-propylene glycol. Other plasticizers which have proved to be suitable for enhancing the elasticity of the films formed from acrylic films such as Eudragit® RL/RS lacquer solutions include polyethylene glycols, propylene glycol, diethyl phthalate, castor oil, and triacetin. Triethyl citrate is an especially preferred plasticizer for aqueous dispersions of ethyl cellulose. It has further been found that addition of a small amount of talc reduces the tendency of the aqueous dispersion to stick during processing and acts a polishing agent.

One commercially available aqueous dispersion of ethylcellulose is Aquacoat® which is prepared by dissolving the ethylcellulose in a water-immiscible organic solvent and then emulsifying the ethylcellulose in water in the presence of a surfactant and a stabilizer. After homogenization to generate submicron droplets, the organic solvent is evaporated under vacuum to form a pseudolatex. The plasticizer is not incorporated into the pseudolatex during the manufacturing phase. Thus, prior to using the pseudolatex as a coating, the Aquacoat® is mixed with a suitable plasticizer.

Another aqueous dispersion of ethylcellulose is commercially available as Surelease® (Colorcon, Inc., West Point, Pa., USA). This product is prepared by incorporating plasticizer into the dispersion during the manufacturing process. A hot melt of a polymer, plasticizer (dibutyl sebacate), and stabilizer (oleic acid) is prepared as a homogeneous mixture which is then diluted with an alkaline solution to obtain an aqueous dispersion which can be applied directly onto substrates.

In one preferred embodiment, the acrylic coating is an acrylic resin lacquer used in the form of an aqueous dispersion, such as that which is commercially available from Rohm Pharma under the trade name Eudragit®. In additional preferred embodiments, the acrylic coating comprises a mixture of two acrylic resin lacquers commercially available from Rohm Pharma under the trade names Eudragit® RL 30 D and Eudragit® RS 30 D. Eudragit® RL 30 D and Eudragit® RS 30 are copolymers of acrylic and methacrylic esters with a low content of quaternary ammonium groups, the molar ratio of ammonium groups to the remaining neutral (meth)acrylic esters being 1:20 in Eudragit® RL 30 and 1:40 in Eudragit® RS 30 D. The mean molecular weight is about 150,000 Daltons. The code designations RL (high permeability) and RS (low permeability) refer to the permeability properties of these agents. Eudragit® RL/RS mixtures are insoluble in water and in digestive fluids, however, coatings formed from them are swellable and permeable in aqueous solutions and digestive fluids.

The Eudragit® RL/RS dispersions may be mixed together in any desired ratio in order to ultimately obtain a controlled release formulation having a desirable dissolution profile. Desirable controlled release formulations may be obtained, for instance, from a retardant coating derived from one of a variety of coating combinations, such as 100% Eudragit® RL; 50% Eudragit® RL and 50% Eudragit® RS; or 10% Eudragit® RL and Eudragit® 90% RS. One skilled in the art should recognize that other acrylic polymers may also be used, for example, Eudragit® L. In addition to modifying the dissolution profile by altering the relative amounts of different acrylic resin lacquers, the dissolution profile of the ultimate product may also be modified, for example, by increasing or decreasing the thickness of the retardant coating.

In preferred embodiments of the present invention, the stabilized product is obtained by subjecting the coated substrate to oven curing at a temperature above the T_{g} of the plasticized acrylic polymer for the required time period, the optimum values for temperature and time for the particular formulation being determined experimentally. In certain embodiments of the present invention, the stabilized product is obtained via an oven curing conducted at a temperature of about 45° C. for a time period from about 1 to about 48 hours. It is also contemplated that certain products coated with the controlled release coating of the present invention may require a curing time longer than 24 to 48 hours, e.g., from about 48 to about 60 hours or more.

The coating solutions preferably contain, in addition to the film-former, plasticizer, and solvent system (i.e., water), a colorant to provide elegance and product distinction. Color may be added to the solution of the therapeutically active agent instead of, or in addition to the aqueous dispersion of hydrophobic material. For example, color may be added to Aquacoat® via the use of alcohol or propylene glycol based color dispersions, milled aluminum lakes and opacifiers such as titanium dioxide by adding color with shear to the water soluble polymer solution and then using low shear to the plasticized Aquacoat®. Alternatively, any suitable method of providing color to the formulations of the present invention may be used. Suitable ingredients for providing color to the formulation when an aqueous dispersion of an acrylic polymer is used include titanium dioxide and color pigments, such as iron oxide pigments. The incorporation of pigments, may, however, increase the retardant effect of the coating.

Spheroids or beads coated with the therapeutically active agents can be prepared, for example, by dissolving the therapeutically active agents in water and then spraying the solution onto a substrate, for example, non pareil 18/20 beads, using a Wuster insert. Optionally, additional ingredients are also added prior to coating the beads in order to assist the binding of the active agents to the beads, and/or to color the solution, etc. For example, a product that includes hydroxypropyl methylcellulose with or without colorant (e.g., Opadry®, commercially available from Colorcon, Inc.) may be added to the solution and the solution mixed (e.g., for about 1 hour) prior to application onto the beads. The resultant coated substrate, beads in this example, may then be optionally overcoated with a barrier agent to separate the therapeutically active agent from the hydrophobic controlled release coating. An example of a suitable barrier agent is one that comprises hydroxypropylmethylcellulose. However, any film-former known in the art may be used. It is preferred that the barrier agent does not affect the dissolution rate of the final product.

Immediate release particles according to the present invention may be coated with a controlled release coating in order to change the release rate to obtain the dissolution rates according to the present invention.

Press Coated, Pulsatile Dosage Form. In another embodiment of the present invention, baclofen is administered via a press coated pulsatile drug delivery system suitable for oral administration with a controlled release component, which contains a compressed blend of an active agent and one or more polymers, substantially enveloped by an immediate release component, which contains a compressed blend of the active agent and hydrophilic and hydrophobic polymers. The immediate release component preferably comprises a compressed blend of active agent and one or more polymers with disintegration characteristics such that the polymers disintegrate rapidly upon exposure to the aqueous medium.

The controlled release component preferably comprises a combination of hydrophilic and hydrophobic polymers. In this embodiment, once administered, the hydrophilic polymer dissolves away to weaken the structure of the controlled release component, and the hydrophobic polymer retards the water penetration and helps to maintain the shape of the drug delivery system.

In accordance with the present invention, the term “polymer” includes single or multiple polymeric substances, which can swell, gel, degrade or erode on contact with an aqueous environment (e.g., water). Examples include alginic acid, carboxymethylcellulose calcium, carboxymethylcellulose sodium, colloidal silicon dioxide, croscarmellose sodium, crospovidone, guar gum, magnesium aluminum silicate, methylcellulose, microcrystalline cellulose, polacrilin potassium, powdered cellulose, pregelatinized starch, sodium alginate, sodium starch glycolate, starch, ethylcellulose, gelatin, hydroxyethyl cellulose, hydroxypropyl cellulose, hydroxypropyl methylcellulose, polymethacrylates, povidone, pregelatinized starch, shellac, zein, and combinations thereof.

The term “hydrophilic polymers” as used herein includes one or more of carboxymethylcellulose, natural gums such as guar gum or gum acacia, gum tragacanth, or gum xanthan, hydroxyethyl cellulose, hydroxypropyl cellulose, hydroxypropyl methylcellulose, methylcellulose, and povidone, of which hydroxypropyl methylcellulose is further preferred. The term “hydrophilic polymers” can also include sodium carboxymethycellulose, hydroxymethyl cellulose, polyethelene oxide, hydroxyethyl methyl cellulose, carboxypolymethylene, polyethelene glycol, alginic acid, gelatin, polyvinyl alcohol, polyvinylpyrrolidones, polyacrylamides, polymethacrylamides, polyphosphazines, polyoxazolidines, poly(hydroxyalkylcarboxylic acids), an alkali metal or alkaline earth metal, carageenate alginates, ammonium alginate, sodium alganate, or mixtures thereof.

The “hydrophobic polymer” of the drug delivery system can be any hydrophobic polymer which will achieve the goals of the present invention including, but not limited to, one or more polymers selected from carbomer, carnauba wax, ethylcellulose, glyceryl palmitostearate, hydrogenated castor oil, hydrogenated vegetable oil type 1, microcrystalline wax, polacrilin potassium, polyethylene oxide, polymethacrylates, or stearic acid, of which hydrogenated vegetable oil type 1 is preferred. Hydrophobic polymers can include, for example, a pharmaceutically acceptable acrylic polymer, including, but not limited to, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methyl methacrylate) copolymer, polyacrylamide, aminoalkyl methacrylate copolymer, poly(methacrylic acid anhydride), and glycidyl methacrylate copolymers. Additionally, the acrylic polymers may be cationic, anionic, or non-ionic polymers and may be acrylates, methacrylates, formed of methacrylic acid or methacrylic acid esters. The polymers may also be pH dependent.

In one embodiment, the delayed or delayed-sustained release coating is an enteric coating. All commercially available pH-sensitive polymers may be used to form the enteric coating. The drug coated with the enteric coating is minimally or not released in the acidic stomach environment of approximately below pH 4.5, but not limited to this value. The drug should become available when the enteric layer dissolves at the higher pH; after a suitable delayed time; or after the unit passes through the stomach. The preferred duration of drug release time is in the range of up to 7 hours after dosing under fasting conditions.

Enteric polymers include cellulose acetate phthalate, cellulose acetate trimellitate, hydroxypropyl methylcellulose phthalate, polyvinyl acetate phthalate, carboxymethylethylcellulose, co-polymerized methacrylic acid/methacrylic acid methyl esters such as, for instance, materials known under the trade name Eudragit® L12.5, Eudragit® L100, or Eudragit® S12.5, S100 (Röhm GmbH, Darmstadt, Germany) or similar compounds used to obtain enteric coatings. Aqueous colloidal polymer dispersions or re-dispersions can be also applied, e.g., Eudragit® L 30D-55, Eudragit® L100-55, Eudragit® S100, Eudragit® preparation 4110D c; Aquateric®, Aquacoat® CPD 30 (FMC Corp.); Kollicoat MAE® 30D and Kollicoat MAE® 30DP (BASF); Eastacryl® 30D (Eastman Chemical, Kingsport, Tenn.).

The enteric polymers used in this invention can be modified by mixing with other known coating products that are not pH sensitive. Examples of such coating products include the neutral methacrylic acid esters with a small portion of trimethylammonioethyl methacrylate chloride, sold currently under the trade names E Eudragit®, Eudragit® RL, Eudragit® RS; a neutral ester dispersion without any functional groups, sold under the trade names Eudragit® NE30D and Eudragit® NE30; and other pH independent coating products.

The term “substantially envelop” is intended to define the total or near-total enclosure of a component. Such an enclosure includes, preferably, at least about 80% enclosure, more preferably at least about 90% enclosure, and even more preferably at least about 99% enclosure.

An embodiment of the present invention provides for a free flowing formulation comprising baclofen. The term “free flowing” as used herein, means dosage forms that pass through a patient's digestive system without impediment or mechanism to slow passage. Thus, for example, the term “free flowing” would exclude gastric raft type dosage forms, which are designed to reside in the stomach for extended periods as in, e.g., U.S. Pat. No. 5,651,985.

Dosage forms according to the present invention can also include a combination of baclofen and at least one additional active agents, such as tizanidine, dantrolene, nonsteroidal anti-inflammatory agents (NSAIDs), opioids, and COX-2 inhibitors. The other active agents can be co-formulated in the immediate-release or delayed-release, delayed-sustained release, or sustained-release components to provide desirable therapeutic effects.

Dosage forms according to the present invention can also apply to pure racemic, L-baclofen, and other GABA related active agents as referred to in U.S. Pat. No. 6,350,769, issued Feb. 26, 2002 to Kaufman et al.

Dosage levels of baclofen (racemic or L-baclofen), as well as any active agent that is to be used in combination with baclofen, in the compositions may be varied so as to obtain an amount of baclofen, and when used as a combination product, active ingredient, that is effective to obtain a desired therapeutic response for a particular composition and method of administration.

An object of the present invention provides for controlled bioavailability of baclofen as desired by health providers. Bioavailability refers to the degree to which the therapeutically active medicament becomes available in the body after administration. Typically, bioavailability is measured in patients who fasted overnight before being dosed with the test preparation. Plasma samples are then taken and analyzed for the plasma concentration of the parent compound and/or its active metabolite. These data may be expressed as C_{MAX}, the maximum amount of active ingredient found in the plasma, or as AUC, the area under the plasma concentration time curve. Shargel & Yu, APPLIED BIOPHARMACEUTICS AND PHARMACOKINETICS ch. 10 (3d ed. 1996); see also APPLIED PHARMACOKINETICS: PRINCIPLES OF THERAPEUTIC DRUG MONITORING, Evans et al., eds. (3d ed. 1992).

For example, baclofen formulations may be used in a comparative bioavailability study in subjects. Patients fast over night prior to drug administration. Plasma samples are then taken at dosing, and every hour for twelve hours after dosing, and then at sixteen and twenty-four hours after dosing, and analyzed for the ng/ml concentration of baclofen or a baclofen metabolite.

Dosage units for rectal administration may be prepared (i) in the form of suppositories which contain the active substance mixed with a neutral fat base; (ii) in the form of a gelatin rectal capsule which contains the active substance in a mixture with a vegetable oil, paraffin oil or other suitable vehicle for gelatin rectal capsules; (iii) in the form of a ready-made micro enema; or (iv) in the form of a dry micro enema formulation to be reconstituted in a suitable solvent just prior to administration.

Liquid preparations for oral administration may be prepared in the form of syrups or suspensions, e.g. solutions or suspensions containing from 0.2% to 20% by weight of the active ingredient and the remainder consisting of sugar or sugar alcohols and a mixture of ethanol, water, glycerol, propylene glycol and polyethylene glycol. If desired, such liquid preparations may contain coloring agents, flavoring agents, saccharin and carboxymethyl cellulose or other thickening agents. Liquid preparations for oral administration may also be prepared in the form of a dry powder to be reconstituted with a suitable solvent prior to use.

Without further elaboration, one skilled in the art having the benefit of the preceding description can utilize the present invention to the fullest extent. The following examples are illustrative only and do not limit the remainder of the disclosure in any way.

### EXAMPLES

### Example 1

### Active Baclofen-Coated Seeds

FORMULATION

| INGREDIENT | % | mg |
|---|---|---|
| Sugar Spheres, NF (mesh 20-25) | 81.4 | 250.0 |
| Micronized Baclofen, USP | 13.0 | 40.0 |
| Povidone, USP (Plasdone K-29/32) | 5.6 | 17.14 |
| Purified Water, USP | N/A | N/A |
| TOTAL: | 100.0 | 307.14 |

Povidone (Plasdone K-29/32®) is added to purified water and mixed until the povidone is fully dissolved. Baclofen is mixed in the above solution until uniformly dispersed. A fluidized bed coating apparatus is then used to coat the sugar spheres with the baclofen suspension to produce active coated seeds.

### Example 2

### Active Baclofen-Coated Seeds

FORMULATION

| INGREDIENT | % | mg |
|---|---|---|
| Sugar Spheres, NF (mesh 20-25) | 81.4 | 250.0 |
| Micronized Baclofen, USP | 13.0 | 40.0 |
| Hypromellose, Type 2910, USP | 5.6 | 17.14 |
| (Pharmacoat 606, 6 cps) | | |
| Purified Water, USP | N/A | N/A |
| TOTAL: | 100.0 | 307.14 |

Hypromellose, Type 2910®, USP (Pharmacoat 606, 6cps) is added to a suitable amount of purified water and mixed until the Hypromellose is fully dissolved. Baclofen is mixed in the above solution until uniformly dispersed. A fluidized bed coating apparatus is then used to coat the sugar spheres with the baclofen suspension to produce active coated seeds.

### Example 3

### Active Baclofen-Containing Granules

FORMULATION

| INGREDIENT | % | mg |
|---|---|---|
| Baclofen, USP | 7.4 | 20.0 |
| Pregelatinized Starch, NF | 21.3 | 57.5 |
| (Starch 1500) | | |
| Microcrystalline Cellulose, NF | 70.8 | 191.3 |
| (Avicel PH-102) | | |
| Magnesium Stearate, NF | 0.5 | 1.3 |
| Purified Water, USP | N/A | N/A |
| TOTAL: | 100.0 | 270.1 |

Mix Baclofen, Starch 1500 (pregelatinized starch) and Avicel PH-102 (microcrystalline cellulose). Charge the baclofen mixture into a Hobart mixer and blend to form a uniform mixture. Granulate the mixture with purified water to form a granulate. Dry the granulate in an oven at a temperature of 60° C. to form granules. Screen the granules using a #30 mesh screen. Mix magnesium stearate to form active granules.

### Example 4

### Enteric-Coated Seeds Containing Baclofen

FORMULATION

| INGREDIENT | % | mg |
|---|---|---|
| Active coated seeds | 76.5 | 153.61 |
| (containing 13.02% Baclofen) | | |
| Hypromellose, Type 2910, USP | 8.5 | 17.07 |
| (Pharmacoat 606, 6 cps) | | |
| Hypromellose Phthalate, NF | 13.5 | 27.11 |
| (HPMCP; HP-50) | | |
| Acetyltributyl Citrate, NF | 1.5 | 3.01 |
| Acetone, NF | N/A | N/A |
| Purified Water, USP | N/A | N/A |
| TOTAL: | 100.0 | 200.8 |

Charge Purified Water into a stainless steel container and mix in Hypromellose until completely dissolved. Then charge Purified Water and Acetone into another stainless steel container and then mix in Acetyltributyl Citrate to form an Acetyltributyl Citrate solution. To this add Hypromellose Phthalate to form an enteric coat solution.

Film coat the Baclofen active coated seeds as produced in any of examples 1-3 with the seal coat solution to form sealed baclofen beads. Then film coat the sealed baclofen beads with the enteric coat solution to produce enteric-coated seeds.

### Example 5

### Enteric-Coated Seeds Containing Baclofen.

FORMULATION

| | A | | B | |
|---|---|---|---|---|
| INGREDIENT | % | mg | % | mg |
| Active coated seeds | 90.0 | 149.4 | 90.0 | 149.4 |
| (containing 13.42% Baclofen) | | | | |
| Methacrylic Acid Copolymer Type | 8.0 | 13.28 | — | — |
| A, NF (Eudragit L 100) | | | | |
| Methacrylic Acid Copolymer Type | — | — | 8.0 | 13.28 |
| C, NF (Eudragit L 100-55) | | | | |
| Talc, USP | 1.0 | 1.66 | 1.0 | 1.66 |
| Triethyl Citrate, NF | 1.0 | 1.66 | 1.0 | 1.66 |
| Isopropyl Alcohol, USP | N/A | N/A | N/A | N/A |
| Purified Water, USP | N/A | N/A | N/A | N/A |
| TOTAL: | 100.0 | 166.00 | 100.0 | 166.0 |

Charge Isopropyl Alcohol and Purified Water into a stainless steel container and then mix in Triethyl Citrate. Add in Methacrylic Acid Copolymer Type A, NF (Eudragit L 100) or 13.28 mg Methacrylic Acid Copolymer Type C, NF (Eudragit L 100-55) to form a Eudragit suspension. Disperse talc into the Eudragit suspension. Film coat the Baclofen active coated seeds from example 4 with the Eudragit suspension to form enteric-coated seeds.

### Example 6

### Composition Containing Baclofen Active Coated and Enteric-Coated Seeds

FORMULATION

| | Immediate release | | Delayed release | | | |
|---|---|---|---|---|---|---|
| Ingredient | component | | component | | TOTAL | |
| Baclofen | 10 | mg | 20 | mg | 30 | mg |
| Pharmacoat 606 | 2 | mg | 4 | mg | 6 | mg |
| Talc | 0.4 | mg | 12.1 | mg | 12.5 | mg |
| Sugar Spheres | 62.5 | mg | 125 | mg | 187.5 | mg |
| Eudragit L100-55 | 0 | | 22.32 | mg | 22.32 | mg |
| Triethyl Citrate | 0 | | 3.72 | mg | 3.72 | mg |
| Water | N/A | | N/A | | N/A | |
| Isopropyl Alcohol | N/A | | N/A | | N/A | |
| Acetone | N/A | | N/A | | N/A | |
| TOTAL: | 74.9 | | 187.14 | | 262.04 | |

Designated portions of active coated seeds and enteric-coated seeds are mixed together to form dosage forms. In the case of capsules the seeds are mixed and added to gelatin capsules. In the case of tablets the seeds are compressed to form a tablet. In the case of sachets, the seed are mixed and filled into the pouch.

### Example 7

### Enteric-Coated Seeds Containing Baclofen

FORMULATION

| INGREDIENT | Weight % |
|---|---|
| Baclofen | 10.56 |
| Sugar Spheres | 65.97 |
| Pharmacoat 606 | 4.52 |
| Eudragit RL 100 | 0.60 |
| Eudragit RS 100 | 1.39 |
| Dibutyl Sebacate | 0.20 |
| Talc | 1.39 |
| Magnesium Stearate | 0.40 |
| HPMCP HP-50 | 13.50 |
| Triethyl Citrate | 1.50 |
| TOTAL: | 100.00 |

Pharmacoat 606 is dissolved in purified water and Baclofen is then dispersed into this aqueous solution to make an aqueous suspension. A fluidized bed coating equipment is used to coat the sugar sphere with the baclofen suspension to produce active coated seeds.

Eudragit RL100, RS 100, and dibutyl sebacate are dissolved in a mixture of acetone and isopropyl alcohol. Talc and magnesium stearate are then dispersed into the solution. A fluidized bed coating equipment is used to coat the active coated seeds with the above suspension to produce sustained-release coated seeds.

HPMCP and triethyl citrate are dissolved in a mixture of acetone and purified water. A fluidized bed coating equipment is used to coat the sustained-release coated seeds with the above solution to produce enteric-coated seeds.

### Example 8

### Baclofen Tablets.

FORMULATION

| INGREDIENT | Weight (mg) |
|---|---|
| Baclofen | 20 |
| Sodium Starch Glycolate | 20 |
| Dicalcium Phosphate Anhydrous | 26.5 |
| Lactose Anhydrous | 132.5 |
| Mg stearate | 1 |
| TOTAL: | 200 |

Mix Baclofen, Sodium Starch Glycolate, Dicalcium Phosphate Anhydrous, and Lactose anhydrous in a high-shear granulator. Wet-Granulate the mixture with purified and dry the granulates in an oven at a temperature of 60° C. for at least 16 hours. Screen the granules using a #25 mesh screen. Mill the oversized granules by a Fitzpatric comminuting machine equipped with a 18 mesh screen. Blend the screened and milled granules with Magnesium Stearate and compress the blend into tablets using a rotary tablet press.

### Example 9

### Baclofen Tablets.

FORMULATION

| INGREDIENT | Weight (mg) |
|---|---|
| Baclofen | 20 |
| Hydroxypropyl Methylcellulose, type | 60 |
| 2910, USP (Methocel K100LV) | |
| Lactose Monohydrate or Mannitol | 39.60 |
| Microcrystalline Cellulose, NF (Avicel | 79.40 |
| PH101 | |
| Magnesium stearate | 1.00 |
| TOTAL: | 200 |

Mix Baclofen, Hydroxypropyl Methylcellulose, Lactose Monohydrate or Mannitol, and Microcrystalline Cellulose in a high-shear granulator. Wet-Granulate the mixture with purified and dry the granulates in an oven at a temperature of 60° C. for at least 16 hours. Screen the granules using a #25 mesh screen. Mill the oversized granules by a Fitzpatric comminuting machine equipped with a 18 mesh screen. Blend the screened and milled granules with Magnesium Stearate and compress the blend into tablets using a rotary tablet press.

### Example 11

### Determining Plasma Profiles for Baclofen-Containing Formulations

At least 12 healthy adult male and female subjects are selected for study. Baclofen is administered orally with 240 ml of room temperature water after subjects are fasted overnight for at least 10 hours. No fluid, except that given with drug administration, is allowed from 1 hour prior to dose administration until 1 hour after dosing. At 2 hours post-dose, subjects will consume 240 ml of room temperature water. Blood samples are drawn at 0.5, 1, 1.5, 2, 2.5, 3, 3.5, 4, 4.5, 5, 6, 7, 8, 10, 12, 16, and 24 hours after administration. The mean plasma concentration-time profile, C_{MAX}, T_{MAX}, C_{MIN}, and T_{MIN} are determined.

Having now fully described this invention, it will be understood to those of ordinary skill in the art that the methods of the present invention can be carried out with a wide and equivalent range of conditions, formulations, and other parameters without departing from the scope of the invention or any embodiments thereof.

1. A pharmaceutical dosage form comprising an immediate release and a controlled release component,
wherein said immediate release component and said controlled release component each comprises a GABA_{B} agonist and a pharmaceutically acceptable excipient;
wherein said immediate release component exhibits an in vitro dissolution profile comprising at least about 80% GABA_{B} agonist release after 1 hour;
wherein said controlled release component exhibits an in vitro dissolution profile in simulated intestinal fluid medium comprising at least about 5% GABA_{B} agonist release after 1 hour, at least about 20% GABA_{B} agonist release after 4 hours, and at least about 30% GABA_{B} agonist release after 6 hours; and
wherein the ratio of said immediate release component to said controlled release component is from about 1:10 to about 10:1.

2. A pharmaceutical dosage form according to claim 1 wherein said ratio of immediate release component to controlled release component is from about 1:4 to about 4:1.

3. A pharmaceutical dosage form according to claim 1 wherein said ratio of immediate release component to controlled release component is from about 1:2 to about 1:1.

4. A pharmaceutical dosage form according to claim 1 wherein said GABA_{B} agonist is baclofen, a baclofen prodrug, a baclofen analog, or a mixture thereof.

5. A pharmaceutical dosage form according to claim 4 wherein said baclofen is a racemic mixture.

6. A pharmaceutical dosage form according to claim 4 wherein said baclofen consists essentially of the L-baclofen enantiomer.

7. A pharmaceutical dosage form according to claim 4 wherein said baclofen comprises at least about 95% L-baclofen enantiomer.

8. A pharmaceutical dosage form according to claim 4 wherein said baclofen is in the amount from about 2 mg to about 150 mg.

9. A pharmaceutical dosage form according to claim 4 wherein said baclofen is in the amount from about 2.5 mg to about 100 mg.

10. A pharmaceutical dosage form according to claim 1 wherein said dosage form is a tablet.

11. A pharmaceutical dosage form according to claim 1 wherein said dosage form is a capsule.

12. A pharmaceutical dosage form according to claim 11 wherein said capsule further comprises discrete units selected from the group consisting of beads, granules, particles, or a mixture thereof.

13. A pharmaceutical dosage form comprising an immediate release and a controlled release component,
wherein said immediate release component and said controlled release component each comprises a GABA_{B} agonist and a pharmaceutically acceptable excipient;
wherein said immediate release component exhibits an in vitro dissolution profile comprising at least about 80% GABA_{B} agonist release after 1 hour;
wherein said controlled release component exhibits an in vitro dissolution profile in simulated gastric fluid/simulated intestinal fluid (1 hour switchover) medium comprising from about 2% to about 90% GABA_{B} agonist release after 1 hour, at least about 30% GABA_{B} agonist release after 4 hours, and at least about 40% GABA_{B} agonist release after 6 hours; and
wherein the ratio of said immediate release component to said controlled release component is from about 1:10 to about 10:1.

14. A pharmaceutical dosage form according to claim 13 wherein said ratio of immediate release component to controlled release component is from about 1:4 to about 4:1.

15. A pharmaceutical dosage form according to claim 13 wherein said ratio of immediate release component to controlled release component is from about 1:2 to about 1:1.

16. A pharmaceutical dosage form according to claim 13 wherein said GABA_{B} agonist is baclofen, a baclofen prodrug, a baclofen analog, or a mixture thereof.

17. A pharmaceutical dosage form according to claim 16 wherein said baclofen is a racemic mixture.

18. A pharmaceutical dosage form according to claim 16 wherein said baclofen consists essentially of the L-baclofen enantiomer.

19. A pharmaceutical dosage form according to claim 16 wherein said baclofen comprises at least about 95% L-baclofen enantiomer.

20. A pharmaceutical dosage form according to claim 16 wherein said baclofen is in the amount from about 2 mg to about 150 mg.

21. A pharmaceutical dosage form according to claim 16 wherein said baclofen is in the amount from about 2.5 mg to about 100 mg.

22. A pharmaceutical dosage form according to claim 13 wherein said dosage form is a tablet.

23. A pharmaceutical dosage form according to claim 13 wherein said dosage form is a capsule.

24. A pharmaceutical dosage form according to claim 23 wherein said capsule further comprises discrete units selected from the group consisting of beads, granules, particles, or a mixture thereof.

25. A pharmaceutical dosage form comprising an immediate release and a controlled release component,
wherein said immediate release component and said controlled release component each comprises a GABA_{B} agonist and a pharmaceutically acceptable excipient; and
wherein said dosage form exhibits an in vivo plasma profile comprising mean maximum GABA_{B} agonist release from about 30 minutes to about 7 hours after administration to a fasting patient.

26. A pharmaceutical dosage form according to claim 25 wherein said in vivo plasma profile comprises mean maximum GABA_{B} agonist release from about 1 hour to about 5.5 hours after administration to a fasting patient.

27. A pharmaceutical dosage form according to claim 25 wherein said in vivo plasma profile comprises mean maximum GABA_{B} agonist release from about 90 minutes to about 5.5 hours after administration to a fasting patient.

28. A pharmaceutical dosage form according to claim 25 wherein said in vivo plasma profile comprises mean maximum GABA_{B} agonist release from about 2 hours to about 5.5 hours after administration to a fasting patient.

29. A pharmaceutical dosage form according to claim 25 wherein said ratio of immediate release component to controlled release component is from about 1:10 to about 10:1.

30. A pharmaceutical dosage form according to claim 25 wherein said ratio of immediate release component to controlled release component is from about 1:4 to about 4: 1.

31. A pharmaceutical dosage form according to claim 25 wherein said ratio of immediate release component to controlled release component is from about 1:2 to about 1:1.

32. A pharmaceutical dosage form according to claim 25 wherein said GABA_{B} agonist is baclofen, a baclofen prodrug, a baclofen analog, or a mixture thereof.

33. A pharmaceutical dosage form according to claim 32 wherein said baclofen is a racemic mixture.

34. A pharmaceutical dosage form according to claim 32 wherein said baclofen consists essentially of the L-baclofen enantiomer.

35. A pharmaceutical dosage form according to claim 32 wherein said baclofen comprises at least about 95% L-baclofen enantiomer.

36. A pharmaceutical dosage form according to claim 32 wherein said baclofen is in the amount from about 2 mg to about 150 mg.

37. A pharmaceutical dosage form according to claim 32 wherein said baclofen is in the amount from about 2.5 mg to about 100 mg.

38. A pharmaceutical dosage form according to claim 25 wherein said dosage form is a tablet.

39. A pharmaceutical dosage form according to claim 25 wherein said dosage form is a capsule.

40. A pharmaceutical dosage form according to claim 39 wherein said capsule further comprises discrete units selected from the group consisting of beads, granules, particles, or a mixture thereof.

41. A pharmaceutical dosage form comprising an immediate release and a controlled release component,
wherein said immediate release component and said controlled release component each comprises a GABA_{B} agonist and a pharmaceutically acceptable excipient; and
wherein said dosage form exhibits an in vivo plasma profile comprising at least two hours of sustained GABA_{B} agonist concentrations at greater than therapeutic levels, after about 2 hours following administration to a fasting patient.

42. A pharmaceutical dosage form according to claim 41 wherein said dosage form further comprises from about 5% to about 85% GABA_{B} agonist release in the stomach.

43. A pharmaceutical dosage form according to claim 41 wherein said dosage form further comprises at least about 25% GABA_{B} agonist release in the intestinal tract.

44. A pharmaceutical dosage form according to claim 41 wherein said dosage form further comprises substantially complete GABA_{B} agonist release after about 10 hours following administration to a fasting patient.

45. A pharmaceutical dosage form according to claim 41 wherein said ratio of immediate release component to controlled release component is from about 1:10 to about 10:1.

46. A pharmaceutical dosage form according to claim 41 wherein said ratio of immediate release component to controlled release component is from about 1:4 to about 4:1.

47. A pharmaceutical dosage form according to claim 41 wherein said ratio of immediate release component to controlled release component is from about 1:2 to about 1:1.

48. A pharmaceutical dosage form according to claim 41 wherein said GABA_{B} agonist is baclofen, a baclofen prodrug, a baclofen analog, or a mixture thereof.

49. A pharmaceutical dosage form according to claim 48 wherein said baclofen is a racemic mixture.

50. A pharmaceutical dosage form according to claim 48 wherein said baclofen consists essentially of the L-baclofen enantiomer.

51. A pharmaceutical dosage form according to claim 48 wherein said baclofen comprises at least about 95% L-baclofen enantiomer.

52. A pharmaceutical dosage form according to claim 48 wherein said baclofen is in the amount from about 2 mg to about 150 mg.

53. A pharmaceutical dosage form according to claim 48 wherein said baclofen is in the amount from about 2.5 mg to about 100 mg.

54. A pharmaceutical dosage form according to claim 41 wherein said dosage form is a tablet.

55. A pharmaceutical dosage form according to claim 41 wherein said dosage form is a capsule.

56. A pharmaceutical dosage form according to claim 55 wherein said capsule further comprises discrete units selected from the group consisting of beads, granules, particles, or a mixture thereof.
